(19) United States
(12) Patent Application Publication
(10) Pub. No.: US 2003/0144883 A1
(43) Pub. Date: Jul. 31, 2003

(54) Method for analyzing side effects and interactions of pharmaceuticals

(76) Inventors: Magnus Fagerholm, Bandhagen (SE); Niklas Kvarnstrom, Norrkoping (SE)

Correspondence Address: ROLF FASTH, FASTH LAW OFFICES, 629 E. BOCA RATON ROAD, PHOENIX, AZ, 85022, US

(21) Appl. No.: 10/283,772

(22) Filed: Oct. 29, 2002

Related U.S. Application Data
(60) Provisional application No. 60/353,495, filed on Jan. 30, 2002.

(51) Int. Cl. G06F 17/60; G06F 19/00; G01N 33/48; G01N 33/50; A61B 5/00

(52) U.S. Cl. 705/2; 702/19; 600/300

(57) ABSTRACT

The method is a computer program for analyzing interactions between pharmaceuticals used by a patient by analyzing the prescribed pharmaceutical with a pharmaceutical profile section showing pharmaceuticals used by the patient, a diagnose profile section showing diagnose information about the patient, and an over-sensitivity profile section showing pharmaceuticals and medical substances to which the patient is sensitive. The program automatically issues warnings if the prescribed drug is incompatible with any of the pharmaceuticals or active substances of the pharmaceuticals listed in the pharmaceutical profile or over-sensitivity profile sections. The program also warns about side effects that may interfere with patient activities such as driving, breast feeding, alcohol, etc.

### PRIOR APPLICATION

This application claims priority from U.S. Provisional Patent Application No. 60/353,495; filed Jan. 30, 2002.

### TECHNICAL FIELD

The present invention relates to a method for analyzing side effects of and interactions between pharmaceuticals.

### BACKGROUND INFORMATION AND SUMMARY OF INVENTION

It is not uncommon for people to simultaneously take different pharmaceuticals or drugs for several illnesses. One problem is that the medical profession may prescribe drugs to the patient without having complete knowledge of the patient's current intake of drugs and whether the prescribed drug may negatively interact with the drugs the patient is already taking. The prescribing physician may rely on information from the patient to obtain information about the patient's current use of drugs. This is often unreliable. Even if the prescribing physician is provided with the patient's current intake of drugs, it is difficult for the physician to know how the prescribed drug may interact with the other drugs without extensive research in databases. It is also difficult to know how the side effects of the drugs may adversely affect normal patient activities such as driving, breast feeding, and other common patient activities. It is also difficult to know the correct dosage for each individual patient. There is a need for a more reliable method of safely prescribing pharmaceuticals to patients without having to do extensive research each time a drug is prescribed.

The method of the present invention provides an effective and reliable solution to the above-outlined problems. More particularly, the method is a computer program for analyzing interactions between pharmaceuticals used by a patient by analyzing the prescribed pharmaceutical with a pharmaceutical profile section showing pharmaceuticals used by the patient, a diagnose profile section showing diagnose information about the patient and an over-sensitivity profile section showing pharmaceuticals and medical substances to which the patient is sensitive. The patient data section lists patient specific parameters such as sex, renal function, age, weight, length, body mass-index, body surface-area, etc. These data are used for determining whether a specific drug should be prescribed or not, as well as for determining the correct dosage. It is also possible to include a section for laboratory results that contains specific laboratory results for the patient that are relevant for determining whether a specific drug should be prescribed or not, as well as for determining the correct dosage. It is also possible to include a genetic profile section that contains patient specific genetic test results that are relevant for drug metabolism and drug effect/side effects to determine whether a specific drug should be prescribed or not, as well as for determining the correct dosage. The program automatically issues warnings if the prescribed drug is incompatible with any of the pharmaceuticals or active substances of the pharmaceuticals listed in the pharmaceutical profile or over-sensitivity profile sections. The program automatically issues warnings if the prescribed drug is incompatible with any of the patients diagnosis listed in the diagnose profile section. The program automatically issues warnings if the prescribed drug has been prescribed before and was discontinued for any reason. The program also gives facts about previous dosages used and why the previous drug treatment was discontinued. The program may also automatically issues warnings if the prescribed drug is incompatible with any of the patients genetic test or laboratory results or the laboratory results listed in the genetic profile section and laboratory data section. The program also warns for side effects that may interfere with patient activities such as driving, breast feeding, alcohol, etc.

### BRIEF DESCRIPTION OF THE DRAWINGS

FIG. 1 is a schematic graphical view of a patient data screen of the present invention;

FIG. 2 is a schematic graphical view of a screen with information about patient over-sensitivity towards pharmaceuticals;

FIG. 3 is a schematic graphical view of a screen with information about patient diagnosis;

FIG. 4 is a schematic graphical view of a screen with information about pharmaceutical substances;

FIG. 5 is a schematic graphical view of interactive effects between pharmaceutical substances;

FIG. 6; is a schematic graphical view of side effects of pharmaceuticals;

FIG. 7; is a schematic graphical view of pharmaceutical effects on pregnancies;

FIG. 8 is a schematic graphical view of pharmaceutical effects on breast-feeding;

FIG. 9 is a schematic graphical view of pharmaceuticals effects on driving in traffic;

FIG. 10 is a schematic graphical view of ATC groups of pharmaceuticals;

FIG. 11 is a schematic flow diagram of the system of the present invention; and

FIG. 12 is a schematic view of prohibitive use of pharmaceuticals.

### DETAILED DESCRIPTION

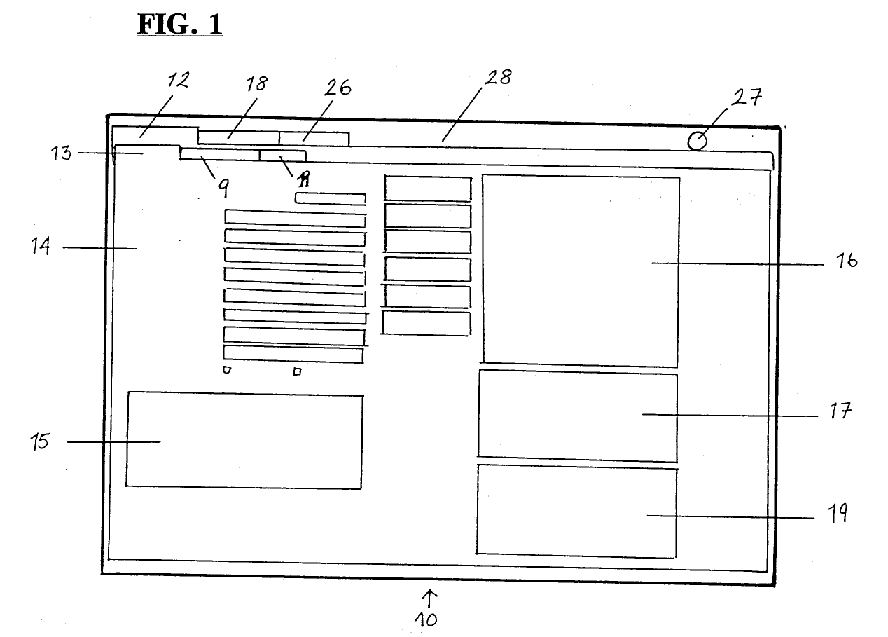

With reference to FIGS. 1-12, the present invention is a unique method for analyzing side effects of and interactions between pharmaceuticals such as prescription drugs. Many patients require, often unnecessarily, treatments due to side effects and undesirable interactions between several drugs prescribed to the patient. An important feature of the present invention is to provide a reliable warning system to reduce the need for such treatments and to give a better overview of the various side effects and interactions that are associated with taking a plurality of medical drugs.

FIG. 1 shows a patient data sheet 12 with basic patient data information 13 including a contact section 14 that, for example, has sections for name, address, telephone number, e-mail address, sex of patient, weight, length, body mass index, body surface area, kidney function, serum creatinine value, and social security number. The patient profile may also include information about the age, liver function, genetic profile, laboratory test-results. For example, the liver and kidney functions affect how quickly substances are eliminated or metabolized by the body. The patient's genetic profile may indicate if a substance is suitable or not. The sheet 12 also has sub-pages such as an over-sensitivity screen 9 and a diagnose screen 11, as discussed in more detail below.

If the patient is not registered in the system, the program may ask for registration of a new patient profile. The patient has the right to have all information removed from the patient database if the patient so desires.

A typical user of the program 10 could be pharmacists and other medical professionals. The sheet 12 also has a pharmaceutical profile section 16 that lists the drugs the patient is currently using. By high lighting one of the drugs and clicking with a mouse device, the user may obtain more detailed information about the particular drug from a database such as a suitable medical database. The sheet 12 may have a free text section 15 where general comments about the patient may be entered that could be useful for future treatments. A diagnose profile section 17 and an over-sensitivity profile section 19 may be disposed below the section 16. The section 17 may include some concise diagnose information about the condition of the patient and the section 19 may include a list of substances and groups of pharmaceuticals to which the patient is particularly sensitive or allergic.

By high lighting one of the diagnoses/over-sensitivity options and clicking with a mouse device, the user may obtain more detailed information and treatment options about the particular diagnosis/over-sensitivity from a medical database. The program may check if any current and future prescribed drugs included in the section 16 is incompatible with the patient's diagnosis in section 17 and issues a warning if such a drug is prescribed. For example, if the diagnosis includes headache and ulcer, the program may warn against the substance for treating the headache if the substance is incompatible with the ulcer and may even worsen the ulcer symptoms. The program may also control whether the prescribed drug is compatible with what is normally prescribed to treat the patient's diagnosis and issues a warning if the prescribed drugs does not match the patient diagnosis. If the prescribed drugs are not compatible, the program may issue a warning. The program may also consider individual characteristics of the patient such as the length, weight, age, sex, kidney and liver functions, genetic profile and match these characteristics against the pharmaceutical and clinical guidelines in the medical database.

The program may also consider the individual dosage of a substance. A suitable dosage size and dosage interval may be calculated based on the patient profile, as listed above, and be matched against clinical guidelines in the medical database.

The program may also check if any substance included in the section 19 is included in current and future prescribed drugs and issues a warning if such a drug is prescribed. If a warning is issued, it may then be possible to identify and subscribe an alternative drug that does not contain the substances to which the patient is allergic or over-sensitive to. The screen 12 gives a good overall view of the condition of and drugs used by a particular patient.

The program 10 also includes a pharmaceutical screen 18, a support/information screen 26 and sub-screens over-sensitivity 9 and diagnose 11, as shown in the tool bar 28 in FIG. 1. The sheet 12 may also have an indicator 27 to show whether a particular patient database has been logged in so that the patient's specific data has been or is being retrieved from the patient database. It is also possible to include the name, social security number, kidney function, body mass index of the logged-in patient. When the user logs out of the patient' database, the user has the option of saving the patient data so that the updated information is displayed next time the user enters the screen 12. The user may, based on the sheet 12, prepare a patient report that shows patient data, pharmaceutical, interactions, diagnosis, over-sensitivity, therapeutic overlap and warnings. The program may also be connected to a magnetic/bar code reader to load patient/pharmaceutical information into the program.

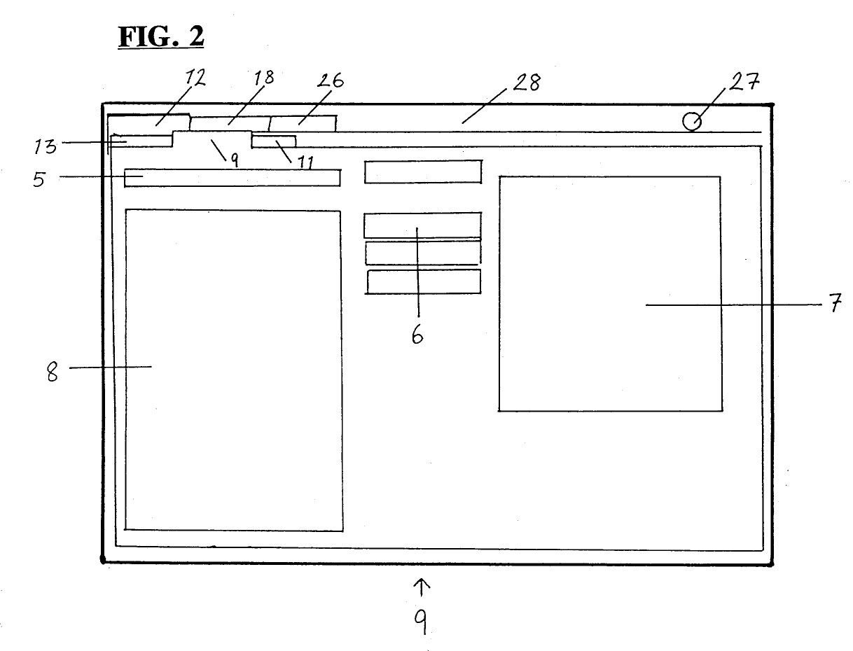

FIG. 2 is a detailed view of the over-sensitivity screen 9 in FIG. 1. The screen 9 has an over-sensitivity screen 8 that list and categorizes over-sensitivity symptoms, according to groups or ATC categories. By high lighting one of the over-sensitivity options and clicking with a mouse device, the user may obtain more detailed information about the particular over-sensitivity and treatment options from a suitable medical database.

The user may select a pharmaceutical or group thereof from the screen 8 and add to the patient's over-sensitivity profile 7 by activating a button 6. Of course, prior selected over-sensitivity may also be removed with activation buttons. The screen 9 also has a search field 5 to search for over-sensitivity descriptions that exist in the screen 8. The profile 7 may be linked to the profile section 19 so that when the profile 7 is updated, the same update appears in the profile section 19, shown in FIG. 1.

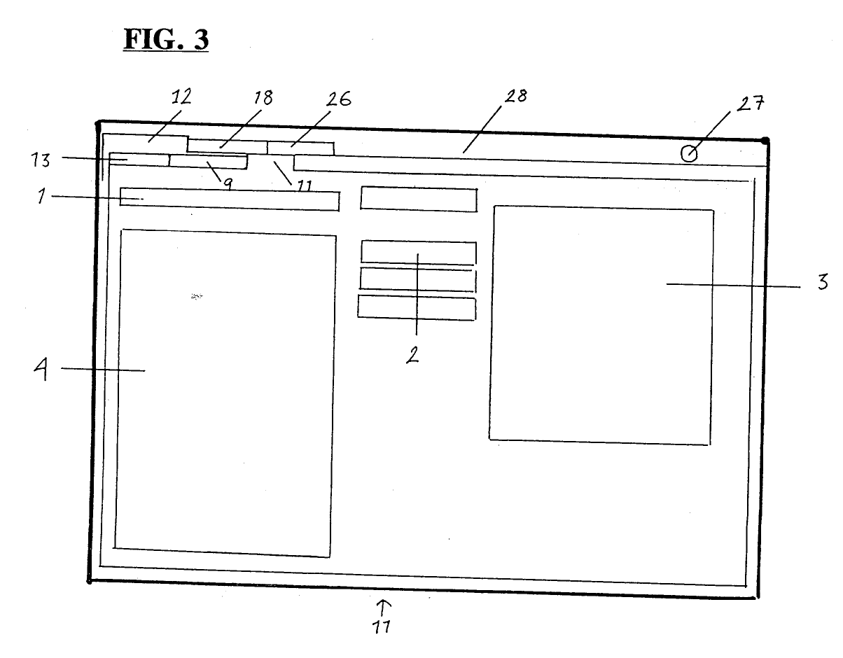

FIG. 3 is a detailed view of the diagnose screen 11 in FIG. 1. The screen 11 has a diagnose screen 4 that lists and categorizes diagnose descriptions according to groups or ICD 10 categories. By high lighting one of the diagnoses and clicking with a mouse device, the user may obtain more detailed information about the particular diagnosis and treatment options from a medical database.

The user may select a diagnosis from the screen 4 and add to the patient's diagnose profile 3 by activating a button 2. Profile information may also be removed from the profile screen 3. The screen 11 also has a search field 1 to search for diagnose descriptions found in the screen 4. Preferably, the profile screen 3 is linked to the profile screen 17, shown in FIG. 1, so that when the profile screen 3 is updated, the profile screen 17 is also automatically updated.

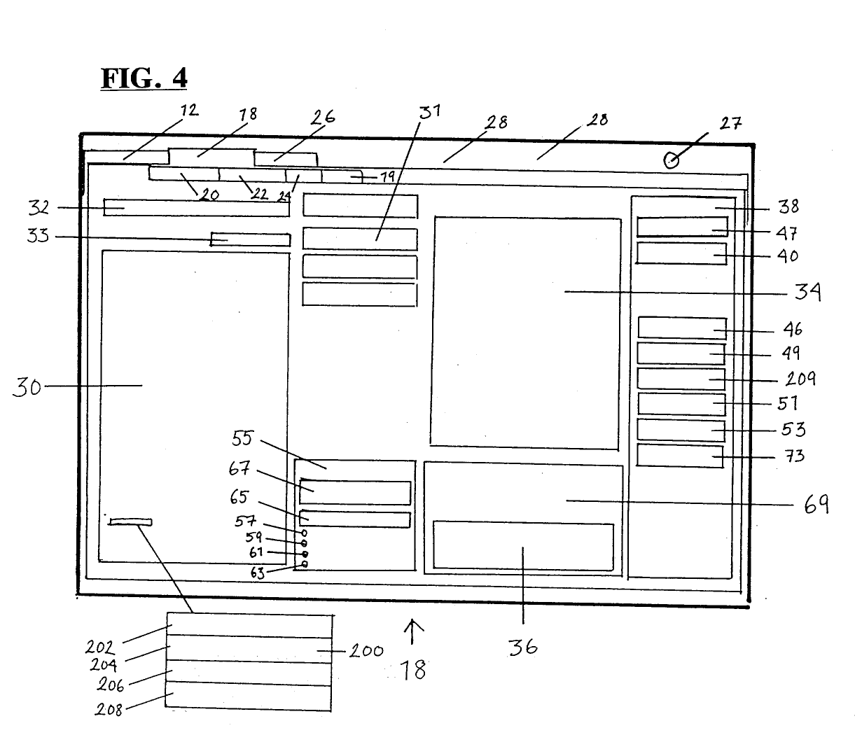

FIG. 4 is a detailed view of the screen button 18 of FIG. 1 that has a pharmaceutical section 30 that lists pharmaceuticals and drugs according to a medical database. By highlighting a drug in the section 30, the user can display a sub-menu 200 that enables the user, by activating an add button 202, to add the identified drug to the profile 34. The user may also activate a show button 204, to show a list of related drugs, activate a patient button 206, to show information about the drug in simple language, and activate a medical button 208, to show information about the drug in more scientific medical language.

The screen 18 has a search section 32 to more conveniently find a drug substance in the section 30. It is also possible to search on a portion of a name of a pharmaceutical. When a drug is prescribed to the patient in question, the drug may be added to a pharmaceutical profile 34 by activating a button 31 or by double clicking on the identified pharmaceutical in the section 30.

Of course, pharmaceutical substances may be removed from the profile 34, as required. By activating a button 33, the drugs in the section 30 are shown in a tree structure. This makes it easier for the user to get an overall view and identify relationships between the drugs. The profile 34 is, preferably, related or linked to the section 16 in FIG. 1 so that when the profile 34 is updated, the same updated information may appear in the section 16. By right clicking on any drug listed in either profile sections 34, 36, the user may obtain more information about the highlighted drug such as related drugs and information from the medical database.

The screen 18 also has a warning section 38 that warns the user if the program 10 identifies undesirable combinations or interactions between the drugs listed in the section 34. If the patient would like to know, for example, which of the substances may affect the patient's driving ability, the user may click on the traffic button 46 to find out which substance on the list 34 should be avoided before driving.

The program may warn about a variety of situations such as undesirable interactions 40, driving in traffic 46, over-sensitivity 47, therapeutic overlap 49, foreign certification requirements 209, pregnancy, breast feeding, doping 51, alcohol 53 and side-effects 75. The program could also include other warnings such as warnings against drug/food interactions, drug/laboratory interference and warnings related to age/gender, drug/disease and drug/sun exposure issues, drug/genetic profile interactions.

By clicking on one of the warning buttons listed below the warning 38, the user may obtain more detailed information about the warning, such as the interaction button 40 or alcohol button 53. The over-sensitivity warning 47 is specific to the particular patient while some of the other warnings may apply to all patients. The doping warning 51 indicates that the substance may affect the performance of an athlete against doping rules and the alcohol warning 53 indicates that the substance should be not combined with alcohol. The side effect warning 75 related to side effects that could be harmful to the patient. Regarding the interaction button 40, there are several types of interactions. Certain interactions are harmful to the body while other undesirable interactions could be that one drug makes another drug ineffective or less effective.

The screen 18 has a related pharmaceutical section 55 that may include a therapeutic main group 57 that is the broadest group. It also includes the slightly narrower therapeutic sub-group 59, chemical/therapeutic sub-group 61 and chemical substance 63. By activating an activation button 67 of a substance marked in the display area 30, the related substances appear in the display area 36. For example, if the substance Magnecyl is entered in the section 65 and the chemical substance 63 is checked, then other drugs with the same chemical active substance and the same ATC code appear in the display area 36. The particular button 57, 59, 61, 63 that is checked and the related ATC code are indicated in a heading section 69 of the display area 36.

In the alternative, if the therapeutic main group 57 is checked, other substances, including the same and different active chemical substances, for treatment of headaches will appear in the area 36. By double clicking on a substance shown in the area 36, the user may add the substance to the profile section 34.

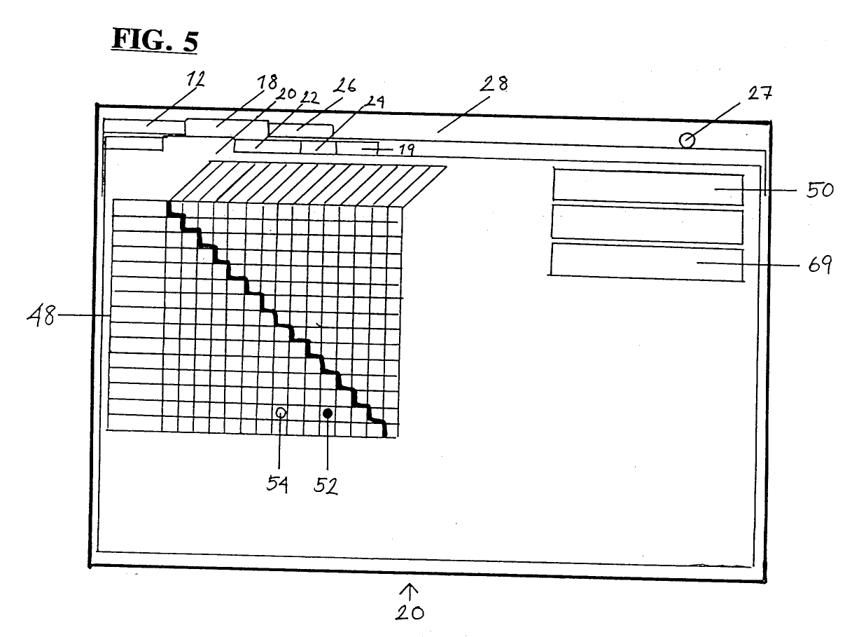

FIG. 5 shows a detailed view of the interaction screen button 20 as shown in FIG. 4. The screen 20 may be displayed by either activating the interaction button 20 in the screen 18 or the interaction warning-button 40 shown in FIG. 4. The screen 20 includes an interaction grid 48 and a grade chart section 50. The grid 48 lists the drugs listed in the profile section 34 and indicates the level of interaction between the drugs. For example, the grid 48 may use grades A, B, C and D. Grade A may symbolize the least severe interaction while Grade D may symbolize the most severe interaction that could lead to severe injury or serious clinical consequences for the patient. For example, in the grid 48, the interaction between the drug Waran and the drug Ipren may be considered a Grade D interaction 52 and should be avoided because it is harmful. On the other hand, the interaction between Waran and Flagyl is a grade C interaction 54 and is not as harmful. It should be noted that problems with interactions could be handled by individual dosages. By dragging the mouse on either the interaction 52, 54, an explanation section 69 appears below the section 50 that explains the harmful interaction between the two substances.

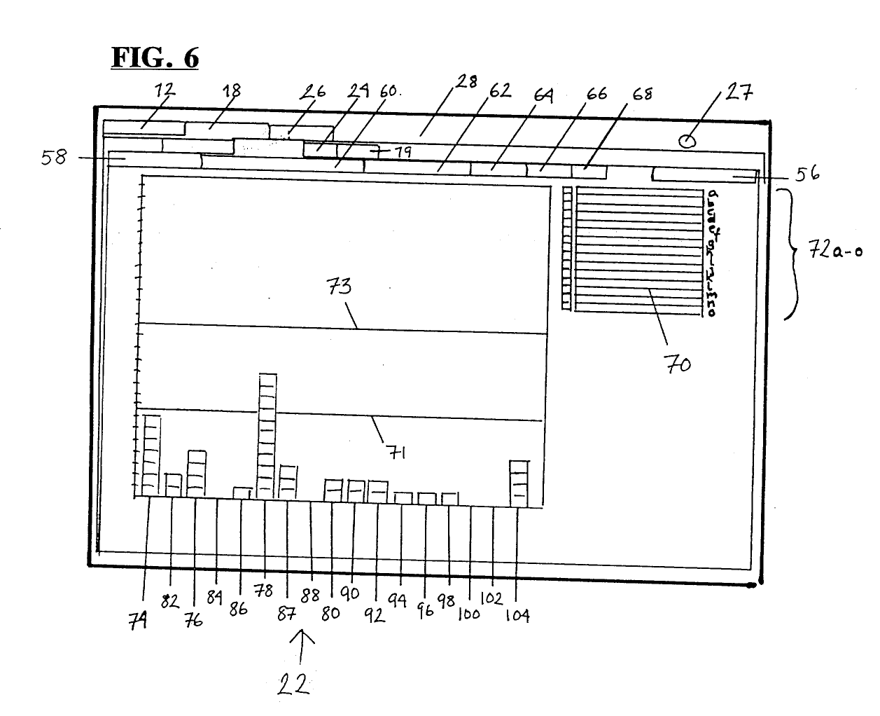

FIG. 6 shows a detailed view of the side effect screen button 22 in screen 18 of FIG. 4. The screen 22 has a bar section 56 at the upper end thereof including the frequency ranges common (more than 1%) 58, less common (between 0.1-1%) 60 and rare (less than 0.1%) 62. The section 56 also has a pregnancy category 64, breast-feeding category 66 and traffic category 68. The categories 64, 66 may be linked to the patient data screen so that they are not shown for male patients and for women who are not in a fertile age. Each drug listed in a drug list 70 that corresponds to the section 16 of FIG. 1 and section 34 of FIG. 2. The side effects of each drug 72a-72o are shown by side effect type so that a cumulative effect of the side effects of all the drugs used by a patient are clearly shown.

For example, the drug 72a may produce the general side effect 74, the blood related side effects 82 and the skin related side effects 87. The side effects of the remaining drugs 72b-o are shown in a similar manner. The other type of side effects may include circulation and heart/vessel side effect 76, muscle related side effects 84, hormonal side effects 86, stomach/intestine related side effects 78, liver related side effects 88, lung related side effect 80, metabolic side effects 90, central nerve system related side effects 92, neurological side effects 94, psychological side effects 96, urine/genital related side effect 98, eye related side effects 100, ear related side effects 102, a miscellaneous side effects 104. In this chart, it can be seen that none of the drugs cause the side effects 84, 88, 100 and 102. By pointing the cursor on one of the boxes on, for example, the side effect 78, the user can see more exactly what the side effects are for each particular drug 72a-72o. It is also possible to see the side effects of a particular drug by double clicking on the name of the drug on the list 70. In this way, all the side effects that are related to the particular drug are marked in the diagram. The screen 22 has a line 71 that indicates the 50% limit of the total number of drugs used by the patient and a line 73 that shows the total number of drugs used by the patient. The side effect warning is issued when the number of side effects in one category on the x-axis exceeds the line 71. Of course, there may be other ways and limits used to trigger the warning. By clicking on the buttons 60, 62, the less common side effects of each drug 72a-72o may be displayed in a manner that is similar to the display of FIG. 6.

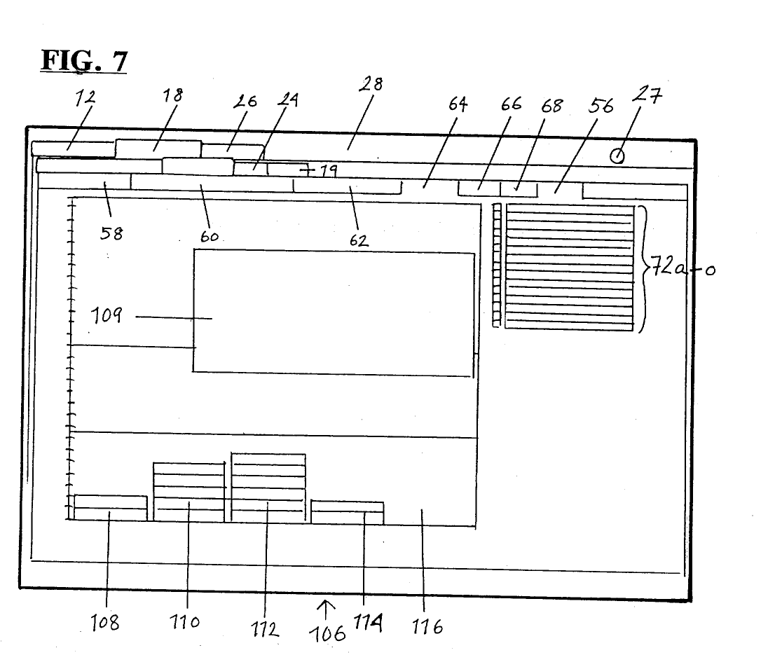

FIG. 7 is a schematic diagram 106 focused on the side effects related to pregnancy 64, as indicated in FIG. 6. The side effects on pregnant women may be categorized as grade A 108, grade B 110, grade C 112, grade D 114 and grade O 116. For example, the drug 72a may cause a grade C pregnancy side effect 112. Grades A and B are generally not harmful to fetus while grades C, D, O are such that a pregnant woman should avoid taking the drug. The cumulative effect of the side effects of each drug 72a-72o is clearly shown.

By placing the cursor on each box, the user may obtain more specific information, as shown in a box 109, about what the side effects are for each drug.

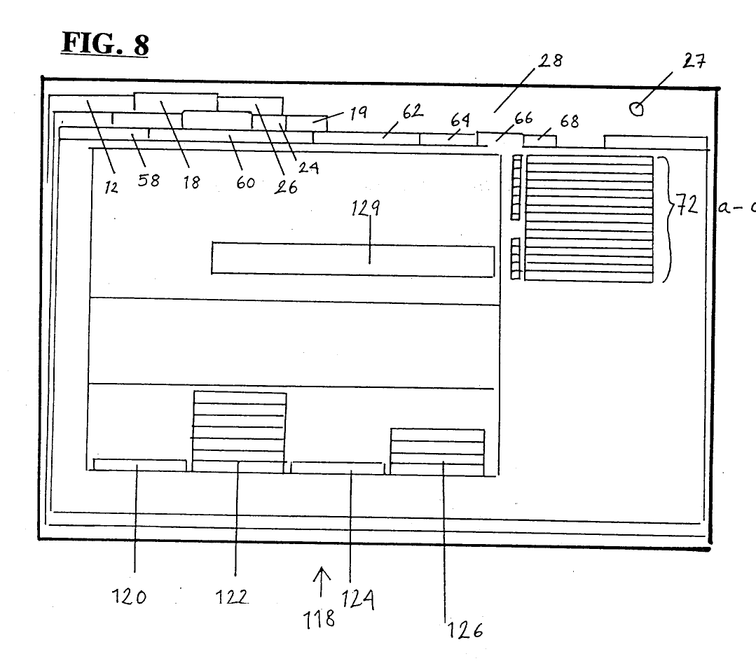

FIG. 8 is a schematic diagram 118 focused on the side effects related to breast feeding 66, as indicated in FIG. 6. The side effects on breast-feeding women may be categorized as category I 120, category II 122, category III 124 and category IV 126. For example, the drug 72c may cause a category II breast-feeding side effect. Categories I/II are generally not harmful while categories III and IV are such that a breast-feeding woman should avoid taking the drug or drugs. For example, category I may be used for substances that does not affect the breast milk while category II is passed to the breast milk but is not harmful to the baby. Category III may indicate that the substance is passed into the breast milk and is harmful to the baby while category IV may be for substances for which insufficient data exists. The cumulative effect of the side effects is shown by category and the specific side effect of each drug may be displayed in a display 129 by placing the cursor on one of the drugs 72a-72o.

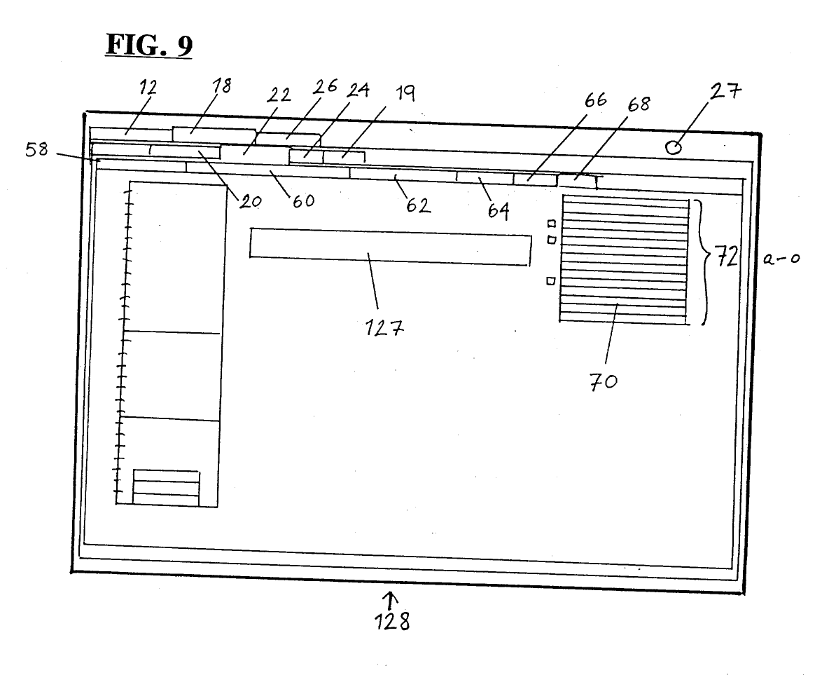

FIG. 9 is a schematic diagram 128 focused on the side effects related to driving in traffic, as indicated by traffic button 68 in FIG. 6. For example, the drug 72f has a negative effect on driving in traffic and should be avoided if the patient intends to drive after taking the drug 72f. The specific side effect section 127 may be displayed by clicking on the box on the X-axis or on the drug in the display list 70.

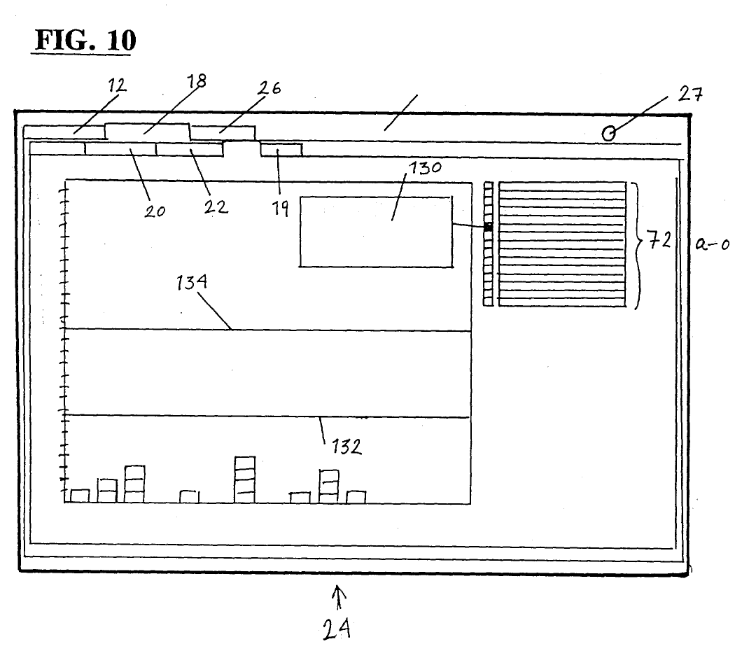

FIG. 10 is a detailed schematic view of the screen 24 of FIG. 4. The drugs 72a-72o are split up into ATC groups A, B, C, D, G, H, J, L, M, N, P, R, S, V according to common practice in the field of pharmaceuticals. Each ATC groups indicate where the body is likely to be affected by the substance. By clicking on, for example, drug 72f an explanation window 130 pops up that details some of the effects of and other information about the drug. By placing the cursor on one of the columns, the program displays more detailed information about each drug regarding the therapeutic groups.

FIG. 11 is a schematic flow diagram 150 of the information flow of the present invention. A user 152, such as a pharmaceutical professional, interacts with the drug interaction program 154. A patient database 156, including specific information, such as over-sensitivity, diagnostic information and pharmaceutical profile information, about the patients, is in communication with the program 154. A medical database 158, including information about pharmaceuticals and their characteristics, is also in communication with the program 154 so that the program 154 may retrieve information from the databases 156, 158, as required. More particularly, the database 158 is connected to an external drug database 160, via a converting data program 162 so that the raw data in the database 160 can be used in the program 154. In other to launch the program 154, a license creator unit 164 may ask for authorization information from the user 152 to make sure that no unauthorized user gain access to the program 154.

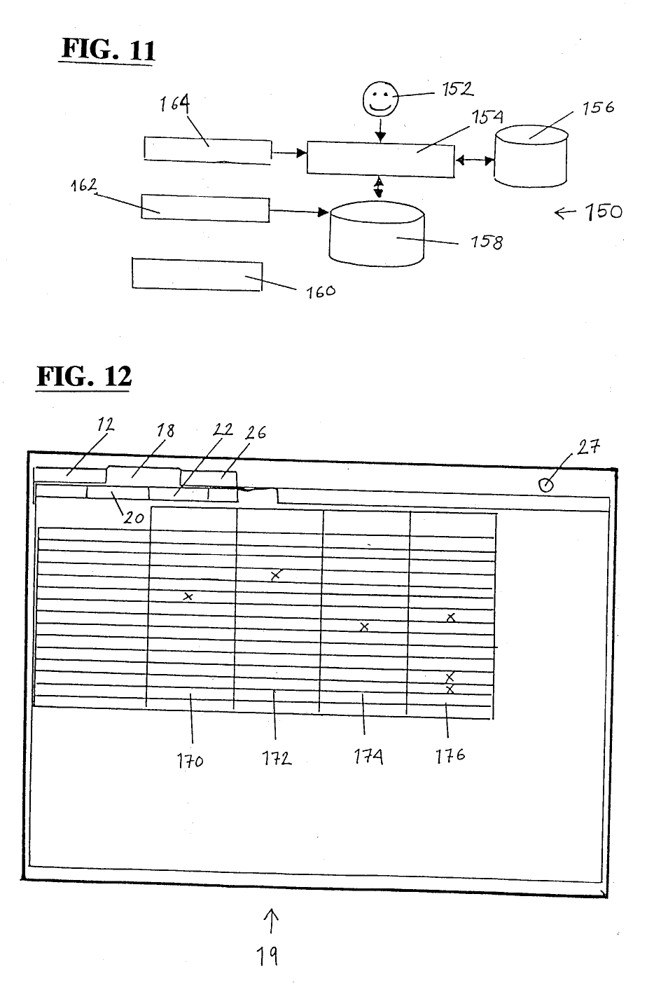

FIG. 12 is a detailed view of the screen 19 that shows prohibitive or disallowed use of the drugs. For example, the column 170 indicates that the international treaties may require certification to bring the drug to foreign countries. The column 172 indicates that the drug may be used for doping. By dragging the mouse over the field, the user may see in more detail which rule applies to certain sport activities. The column 174 shows that the drug must not be combined with alcohol. The user may again obtain more detailed information by dragging the mouse cursor over the field. The column 176 shows the drugs to which the patient is over-sensitive and more detailed information may be obtained by dragging or placing the cursor on the field.

While the present invention has been described in accordance with preferred compositions and embodiments, it is to be understood that certain substitutions and alterations may be made thereto without departing from the spirit and scope of the following claims.

We claim:

1. A method of analyzing interactions between pharmaceuticals used by a patient, comprising:
providing a computer program having patient data information of a patient, a pharmaceutical profile section showing pharmaceuticals used by the patient, a diagnose profile section showing diagnose information about the patient and an over-sensitivity profile section showing pharmaceuticals and medical substances to which the patient is sensitive;
analyzing a medical database linked to the computer program to determine if a prescribed pharmaceutical is incompatible with the diagnose information in the diagnose profile section and issuing a warning when the prescribed pharmaceutical is incompatible with the diagnose information;
analyzing active substances of the prescribed pharmaceutical and determining if the prescribed pharmaceutical is included in the over-sensitivity profile section and issuing a warning when the prescribed pharmaceutical is included or contains a substance that is included in the over-sensitivity profile section; and
determining if the prescribed pharmaceutical is incompatible with the pharmaceuticals of the pharmaceutical profile section and issuing a warning when the prescribed pharmaceutical is incompatible with the pharmaceuticals of the pharmaceutical profile section.

2. The method according to claim 1 wherein the method further comprises analyzing a dosage of the prescribed pharmaceutical based on the patient data information.

3. The method according to claim 1 wherein the method further comprises automatically linking the prescribed pharmaceutical to the medical database containing detailed information about the prescribed pharmaceutical.

4. The method according to claim 1 wherein the method further comprises adding the prescribed drug to the pharmaceutical profile section of the patient when the prescribed drug is not incompatible with the pharmaceuticals listed in the over-sensitivity profile section.

5. The method according to claim 1 wherein the method further comprises listing a set of patient activities and analyzing interactions between the prescribed pharmaceutical and the pharmaceuticals in the pharmaceutical profile section and issuing a warning when the interactions negatively affect at least one of the patient activities.

6. The method according to claim 1 wherein the method further comprises linking the prescribed pharmaceutical to the medical database and listing active substances of the prescribed pharmaceutical.

7. The method according to claim 1 wherein the method further comprises suggesting an alternative pharmaceutical when the prescribed pharmaceutical negatively interacts with the pharmaceuticals in the pharmaceutical profile section to produce side effects.

8. The method according to claim 1 wherein the method further comprises cumulative side effects of the pharmaceuticals of the pharmaceutical profile section.
